(19) United States
(12) Patent Application Publication
(10) Pub. No.: US 2011/0197896 A1
(43) Pub. Date: Aug. 18, 2011

(54) ELASTIC SEAMED THIN-WALLED ARTICLE WITH A SINGLE OPENING

(76) Inventor: Der-Lin LIOU, Taipei City (TW)

(21) Appl. No.: 12/707,524

(22) Filed: Feb. 17, 2010

(51) Int. Cl. A61F 6/04; B29D 22/00; A41D 13/08; A41D 19/00; A63H 3/06; A42B 1/12; A43B 3/16

(52) U.S. Cl. 128/844; 428/35.7; 2/21; 2/167; 446/220; 2/68; 36/7.1 R

(57) ABSTRACT

An elastic seamed thin-walled article having a single opening consists of two halve pair-hand shape films made from thermoplastic material and bonded through a coupling portion. It has an opening formed with a lip portion in an integrated manner. The thin-walled article is formed by at least one layer of the thermoplastic material to provide desired elasticity and recyclable characteristics.

### FIELD OF THE INVENTION

The present invention relates to an elastic seamed thin-walled article with a single opening and particularly to a thin-walled article that has one opening and a lip portion at the opening.

### BACKGROUND OF THE INVENTION

The conventional method of manufacturing elastic thin-walled articles (such as disposable gloves, finger cots, condoms, balloons and the like with an opening at one end) are made of dipping a mold in a liquid state natural or synthetic rubber to form a thin film, then drying to become a finished product. The aforesaid method creates a lot of problems, such as consuming a great amount of water and energy, has to add chemical materials such as sulfur and accelerator that could cause allergy to users. Solvents, chlorine and plasticizers also commonly be used that create environmental pollution and genetic lesion to users. Moreover, most rubber materials are difficult or costly to recycle. There is another method such as U.S. Pat. Nos. 4,776,921, 5,833,915, 5,997,676, 6,243,875 and 6,712,832 to produce thin-walled articles that adopt thermobonding by heat-sealing or heat-welding to bond two layers of thermoplastic material. This method is simpler and less expensive. But the finished product is inelastic and cannot fit snugly when in use. It gives users a poorer tactile feeling. It also does not have an elastic band at the opening, hence easily loosens off when in use.

Thermoplastic elastomer is a recyclable material and has rubber characteristics, and can be processed by traditional plastic manufacturing methods more efficiently. It is also more environmental friendly than the traditional natural—or synthetic rubber. Hence the prevailing trend in the industry is using the thermoplastic elastomer to substitute the traditional rubber.

U.S. Pat. No. 5,640,720 provides an injection blow molding method for manufacturing elastic synthetic rubber gloves from polyetherester block copolymer. The produced gloves have uneven film thickness. U.S. Pat. No. 4,921,672 provides an injection molding method for manufacturing electrical protective gloves. The method can only produce gloves of a thicker film, but not gloves of a thinner film for disposable purpose.

U.S. Pat. Nos. 4,434,126 and 6,890,475 provide a powder coating method for manufacturing thin-film gloves with thermoplastic elastomer. Variation of powder particle size affects film quality and could result in defects such as needle holes or uneven thickness. To produce the particles with the size variation within a small range is costly. Moreover, the thermoplastic elastomer has to be hardened by cooling through a cryogenic process using liquid nitrogen. It is an expensive process.

Thus, based on the issues of raw material cost, recycle possibility and users' health and allergy concern, and other requirements such as high degree of automatic manufacturing, and saving of water and energy, all the conventional techniques mentioned above still leave a lot to be desired. There are still rooms for improvement.

### SUMMARY OF THE INVENTION

In view of the deficiency of the aforesaid conventional techniques that produce seamed thin-walled articles without an integrated lip portion and elasticity, the primary object of the invention is to provide a seamed thin-walled article that is elastic and has a lip portion.

The present invention provides an elastic seamed thin-walled article with a single opening. It consists of two halve pair-hand shape films made from thermoplastic material and bonded through a coupling portion. The thin-walled article has one opening. The opening has a lip portion integrally formed thereon. The thin-walled article consists of at least one layer of thermoplastic material that provides a desired elasticity and recyclable characteristics.

In short, the seamed thin-walled article according to the invention provides many benefits, notably:

1. Using the thermoplastic material that is more environmental friendly and recyclable without irritating human body.

2. Finished products are elastic and can fit snugly when in use.

3. The opening has an integrated lip portion which can be formed in a color different from the article body to facilitate differentiation, fetching and wearing. It is also less likely to loosen off.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 is a plane view of the thin-walled article according to the invention.

FIG. 2 is a side view of the thin-walled article according to FIG. 1.

FIG. 3 is a sectional view according to FIG. 2.

FIG. 4 is an enlarged fragmentary view of a lip portion according to FIG. 3.

FIG. 5 is a cross section taken on line 5-5 in FIG. 3.

FIG. 6 is a plane view of embodiment-1 of the thin-walled article.

FIG. 7 is a plane view of embodiment-2 of the thin-walled article.

FIG. 8 is a plane view of embodiment-3 of the thin-walled article.

### DETAILED DESCRIPTION OF THE PREFERRED EMBODIMENTS

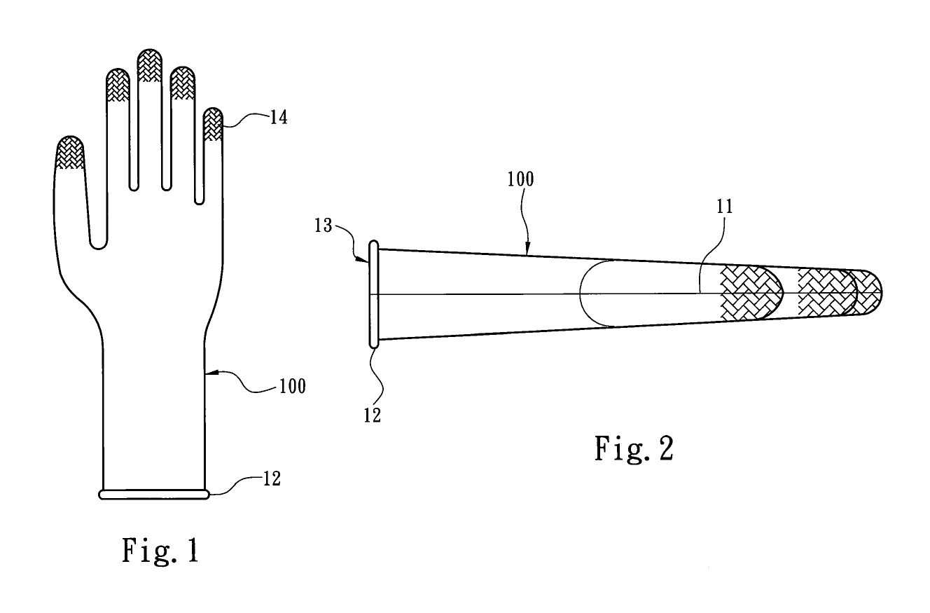

Please refer to FIGS. 1 and 2, the present invention provides an elastic seamed thin-walled article with a single opening. FIG. 1 illustrates the thin-walled article in the form of a glove 100 with a seam and one opening 13. The thin-walled article (namely the glove 100) consists of two halve pair-hand shape films made from thermoplastic material (i.e. an upper half and a lower half of the glove 100) and bonded through a coupling portion 11. The thin-walled article may have embossed traces 14 on the surface (outer surface or inner surface) to increase friction to facilitate grasping, enhance user's tactile feeling or improve product identification. Also referring to FIGS. 2, 3 and 4, the glove 100 has an integrated lip portion 12 at the opening 13 to facilitate user's fetching of the glove 100. The lip portion 12 is elastic to prevent loosening off when put on by users. The lip portion 12 further may be colored to indicate product type, such as large size (Size L) and small size (Size S) by a green and yellow lip portion 12. The thin-walled article may also be formed in varying types and differentiated by the color of the lip portion 12. For instance, the glove 100 with embossed traces 14 and without embossed traces 14 may be differentiated by florescent green and dark green. The above examples illustrate merely possible applications of the lip portion 12. Actual color definitions can be determined by producers.

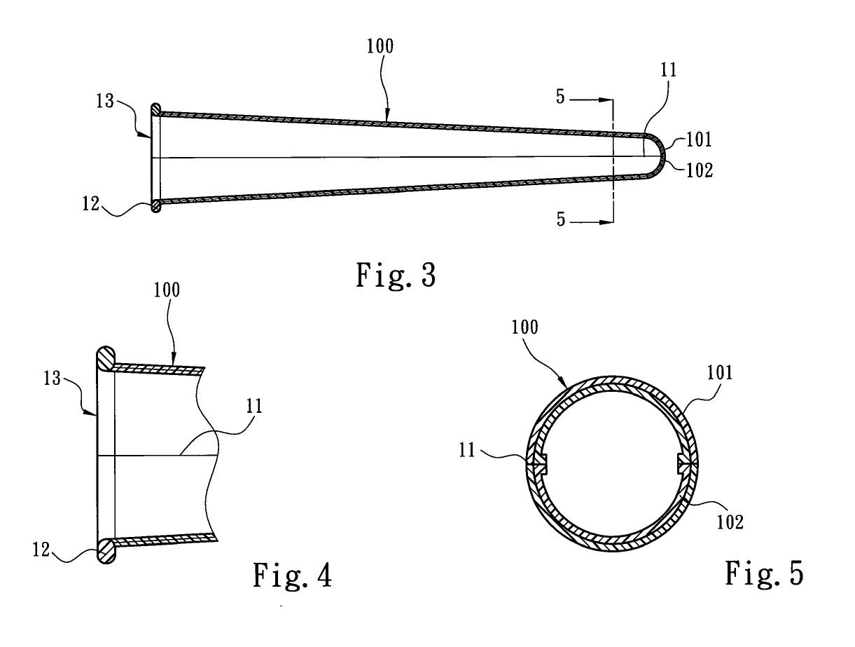

The thin-walled article consists of at least one layer of thermoplastic material. To enhance physical strength or elasticity, or provide the thin-walled article with different frictions on the inner and outer surfaces, the thin-walled article may be formed with multiple layers of the thermoplastic material. Each layer of thermoplastic material has different compositions. Also referring to FIGS. 3, 4 and 5, with FIG. 5 showing a cross section taken on line 5-5 in FIG. 4, the glove 100 has a two-layer structure made from a first thermoplastic material 101 and a second thermoplastic material 102 that have different compositions or densities, or different compositions and densities at the same time. Thus desired physical properties can be derived according to design requirements of the producers. The thermoplastic material previously discussed includes at least 20% thermoplastic elastomer which may be selected from the group consisting of polyamide elastomer, polyurethane elastomer, polystyrene block copolymers and polyolefin elastomer.

In addition to the thermoplastic elastomer, the thermoplastic material may also include polyethylene, polypropylene, antioxidant, stabilizer, anti-blocking agent, mold release agent, filler or color pigments or the like.

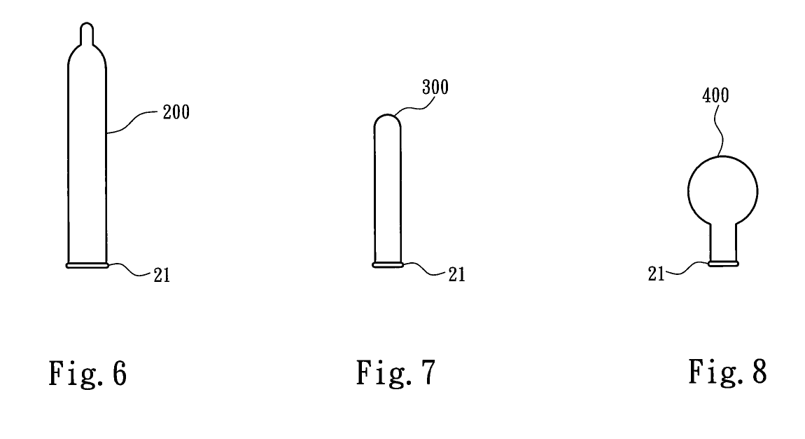

As a conclusion, the thin-walled article according to the invention is elastic and has a single opening with a lip portion formed thereon. The materials used are environmental friendly without irritating human body, and no harmful chemical additives are being used. It provides significant improvements over the conventional techniques. The thin-walled article of the invention is adaptable to various types of products, such as a glove 100 (as shown in FIG. 1), a condom 200 (as shown in FIG. 6), a finger cot 300 (as shown in FIG. 7), a balloon 400 (as shown in FIG. 8), shower caps (not shown in the drawings), or shoe covers (also not shown in the drawings) depending on selections and designs of the producers. As shown in the drawings, the condom 200, finger cot 300 and balloon 400 have respectively a lip portion 21 formed thereon.

While the preferred embodiments of the invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention.

What is claimed is:

1. An elastic seamed thin-walled article having a single opening, comprising:
two halve pair-hand shape films made from thermoplastic material and bonded through a coupling portion and having an opening which has a lip portion formed thereon.

2. The elastic seamed thin-walled article of claim 1, wherein the thin-walled article has embossed traces formed on the surface thereof.

3. The elastic seamed thin-walled article of claim 1, wherein the thin-walled article consists of at least one layer of the thermoplastic material.

4. The elastic seamed thin-walled article of claim 3, wherein the thin-walled article consists of multiple layers of the thermoplastic material, each layer being formed by the thermoplastic material of a different composition.

5. The elastic seamed thin-walled article of claim 1, wherein the lip portion is colored to indicate product types.

6. The elastic seamed thin-walled article of claim 1, wherein the thermoplastic material includes at least 20% thermoplastic elastomer.

7. The elastic seamed thin-walled article of claim 6, wherein the thermoplastic elastomer is polystyrene block copolymer or a mixture thereof.

8. The elastic seamed thin-walled article of claim 6, wherein the thermoplastic elastomer is polyolefin elastomer or a mixture thereof.

9. The elastic seamed thin-walled article of claim 6, wherein the thermoplastic elastomer is polyurethane elastomer or a mixture thereof.

10. The elastic seamed thin-walled article of claim 6, wherein the thermoplastic elastomer is polyamide elastomer or a mixture thereof.

11. The elastic seamed thin-walled article of claim 6, wherein the thermoplastic material further includes polyethylene, polypropylene, antioxidant, stabilizer, anti-blocking agent, mold release agent, filler or color pigments.

12. The elastic seamed thin-walled article of claim 1, wherein the thin-walled article is a glove.

13. The elastic seamed thin-walled article of claim 1, wherein the thin-walled article is a finger cot.

14. The elastic seamed thin-walled article of claim 1, wherein the thin-walled article is a balloon.

15. The elastic seamed thin-walled article of claim 1, wherein the thin-walled article is a condom.

16. The elastic seamed thin-walled article of claim 1, wherein the thin-walled article is a shower cap.

17. The elastic seamed thin-walled article of claim 1, wherein the thin-walled article is a shoe cover.
